(19) United States
(12) Patent Application Publication
(10) Pub. No.: US 2004/0215320 A1
(43) Pub. Date: Oct. 28, 2004

(54) Integral stent graft

(76) Inventor: James Machek, Windsor, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/423,370

(22) Filed: Apr. 25, 2003

(51) Int. Cl. A61F 2/06

(52) U.S. Cl. 623/1.13; 623/1.19; 623/1.53

(57) ABSTRACT

A unitary or integral stent graft having both structural and fluid exclusion properties if formed. The stent graft includes structural elements, such as wires, which are interdigitated with fluid barrier elements, such as a fabric, to form a unitary body such that the graft need not be sewn or otherwise attached to a structural stent member. In one aspect, the stent graft has a shape memory feature, such that is may be manufactured in its final use configuration, distorted for easy delivery to an aneurysmal blood vessel, site and deployed, and in location regain its original shape.

### BACKGROUND OF THE INVENTION

1. Field of the Invention

The present invention relates to the field of lumen repair, more particularly to the field of exclusion devices such as stent grafts placed in body lumens, such as blood vessels, to bypass portions thereof in a diseased or damaged condition, such as that caused by the occurrence of aneurysm.

2. Description of the Related Art

Aneurysms occur in blood vessels in locations where, due to age, disease or genetic predisposition, the blood vessel strength or resiliency is insufficient to enable the blood vessel wall to retain its shape as blood flows therethrough, resulting in a ballooning or stretching of the blood vessel at the limited strength/resiliency location to thereby form an aneurysmal sac. If the aneurysm is left untreated, the blood vessel wall may continue to expand, to the point where the remaining strength of the blood vessel wall is below that necessary to prevent rupture, and the blood vessel will fail at the aneurysm location, often with fatal result.

To prevent rupture of an abdominal aortic aneurysm, a stent graft of a tubular construction is introduced into the blood vessel, such as from a remote location through a catheter introduced into a major blood vessel in the leg and pushed through the blood vessel to the aneurysm location. The stent graft is deployed and secured in a location within the blood vessel such that the stent graft spans the aneurysmal sac. The outer surface of the stent graft, at its opposed ends, is sealed to the interior wall of the blood vessel (aorta) at a location where the blood vessel wall has not suffered a loss of strength or resiliency, such that blood flowing through the vessel is channeled through the hollow interior of the stent graft, and thus reduces, if not eliminates, the stress on the blood vessel wall at the aneurysmal sac location. Therefore, the risk of rupture of the blood vessel wall at the aneurysmal location is significantly reduced, if not eliminated, and blood can continue to flow through to the downstream blood vessels without interruption.

Stent grafts are typically configured by separately forming the graft and the stent, and then attaching the graft to the stent. The graft provides a tubular pathway for blood to flow past the aneurysm, as well as a mechanism to seal off the aneurysmal sac from the blood flow by sealingly engaging the blood vessel wall at the opposed ends thereof. The graft may be manufactured in sheet or tubular form, such as by weaving, knitting or braiding the graft material into a fabric sheet or tube. The stent provides rigidity and structure, to hold the graft open in the tubular shape, as well as to press the graft material into engagement with the blood vessel wall to effectuate the sealing therewith. The stent is typically manufactured by folding or bending individual elements of wire, laser or other cutting of sheets or tubes, or otherwise forming shapes to provide a relatively rigid structure to support the graft.

To attach the graft to the stent, the graft is typically inserted into, or pulled over, the stent, and the graft is sewn to the structural components of the stent. Alternatively, the stent may be formed on the graft such that the individual wires of the stent are threaded though specially provided projecting fabric loops on the surface of the graft, thereby creating attachment of the graft to the stent. The stent and graft are sized such that upon placement thereof into an aneurysmal blood vessel, the diameter of the stent graft slightly exceeds the existing diameter of the blood vessel at healthy blood vessel wall site adjacent to the aneurysm.

The existing stent grafts, as well as the methodologies of their manufacture, result in several drawbacks for both the patient receiving them and the physician or practitioner delivering them. Foremost, the bulk of the stent graft, including the wire cage of the stent and the fabric of the graft, as well as the bulk of the connection mechanisms between them, limits the ultimate size of stent graft that can be made and still fit within a catheter for smaller blood vessel diameter locations. Additionally, the known mechanisms for attachment of the graft to the stent all provide potential sites for separation between the graft and stent, thus providing the risk that the graft may, at least on a localized basis, separate from the stent and sag or bow into the blood vessel. For example, during manufacture, handling or delivery of the stent graft, the attachment mechanisms may tear or fail, allowing the graft to partially or fully separate from the stent.

### SUMMARY OF THE INVENTION

The present invention provides methods and apparatus for the treatment of diseased or damaged lumens, such as blood vessels, as well as methods of manufacture of the apparatus.

In one embodiment, the invention provides an exclusion device having a stent graft structure, wherein the stent and graft are integrally formed such that the stent and graft are formed as a single unitary body. In one aspect, the stent graft includes a graft, formed of a fluid barrier material, within which is formed a stent material, as an integral part thereof. In one embodiment, the stent graft is woven, such that a graft material, formed of fibers, is integrally woven with a stent material, so that a resulting stent graft is formed having the stent integrally provided with the graft. In another embodiment, the stent and graft materials are interbraided, such that individual filaments of the ultimate stent structure are integrally braided with the material forming the graft, such that an integral stent graft is formed.

In a further embodiment, the stent material is a shape memory element, such that, upon formation of the integral stent graft, the stent graft is sized and configured to have an equivalent size and shape to that of the stent graft in use. Once formed, the stent graft is treated to enable compression thereof, such that it may be sized to be introduced into a blood vessel for placement at the affected blood vessel location.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a partial sectional view of a descending aorta, having a bifurcated stent graft configured according to the present invention received therein;

FIG. 2 is a plan view of a portion of the bifurcated stent graft of FIG. 1, showing details of construction thereof,

FIG. 3 is an enlarged view of a portion of the stent graft of FIG. 2, showing the interbraided structure thereof;

FIG. 4 is a plan view of an alternative embodiment of a portion of the stent graft of FIG. 1;

FIG. 5 is an enlarged view of a portion of the portion of the stent graft of FIG. 4.

FIG. 6 is an additional exploded view of a portion of the stent graft of FIG. 4, showing a portion of the stent graft in a partially manufactured state

FIG. 7 is a sectional view of the partial view of the partially assembled portion of a stent graft of FIG. 6 at 7-7

FIG. 8a is an isometric view of a portion of the bifurcated stent graft of the FIG. 1;

FIG. 8b is a plan view of the portion of the stent graft of FIG. 8a, showing the stent graft in a flattened state;

FIG. 8c is an isometric view of the flattened portion of the stent graft of FIG. 8b shown flattened and rolled; and

FIG. 8d is an isometric view of the flattened and rolled stent graft portion of FIG. 8c disposed in a delivery catheter.

### DETAILED DESCRIPTION OF THE PREFERRED EMBODIMENT

A device according to the present invention provides a stent graft for placement to span an aneurysmal blood vessel location, with ease of manufacture and placement thereof. In particular, this configuration eliminates the need to provide separate structure for the stent and graft, enables the creation of customized stent graft for patient placement, provides a smaller material volume stent graft structure, and provides a readily placeable stent graft contoured to the specific attributes of a patient.

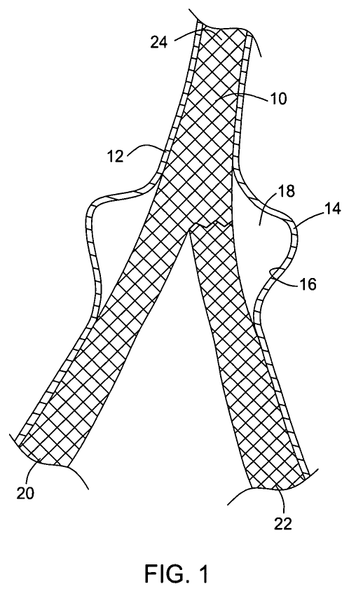

Referring initially to FIG. 1, there is shown an intravascular exclusion or repair vehicle, specifically a bifurcated stent graft 10, positioned in a blood vessel, in this embodiment, an aorta 12, and spanning, within the aorta 12, an aneurysmal portion 14 of the aorta 12. The aneurysmal portion 14 is formed of a bulging of the aorta wall 16, in a location where the strength and resiliency or the aorta wall 16 is weakened. As a result, an aneurysmal sac 18 is formed of distended vessel wall tissue. The stent graft 10 is positioned spanning the sac 18 and thereby provides both a secure passageway for blood flow through the aorta 12 and sealing of the aneurysmal portion 14 of the aorta 12 from additional blood flow from the aorta 12. The placement of the stent graft 10 in the aorta 12 is a technique well known to those skilled in the art, and essentially includes the opening of a blood vessel in the leg, and the insertion of the stent graft 10 contained in a catheter (not shown) into the vessel and through the vessel until deployed to be located in a spanning position across the aneurysmal portion 14 of aorta 12. The bifurcated stent graft 10 has a pair of branched sections 20, 22 bifurcating from a trunk portion 24 thereof. Typically, the bifurcated stent graft has two sections, a first section forming the trunk portion 24 and one of beyond the aneurismal sac. The stent graft is then deployed, i.e., pushed out of the open end of the catheter, as the catheter is withdrawn slowly across the aneurismal sac. This is typically accomplished by holding stationary a rod disposed in the catheter and terminating adjacent to the stent graft received therein, while withdrawing the tubular section of the catheter from the artery such that the trunk portion of the stent graft spans the trunk of the artery and branch section is disposed in one branch of the aorta. The trunk includes a generally circular opening adjacent the extension of branch portion 20 thereof, and a catheter, having branch section 22 therein, is deployed through an artery in the other leg of the patient, such that branch section 22 is deployed therefrom with one end in the aperture and a second end deployed within the other branch of the artery. The procedure, and attachment mechanisms for assembling the stent graft 10in place in this configuration, is well known in the art, and is also disclosed in U.S. Pat. No. 6,203,568, incorporated herein by reference.

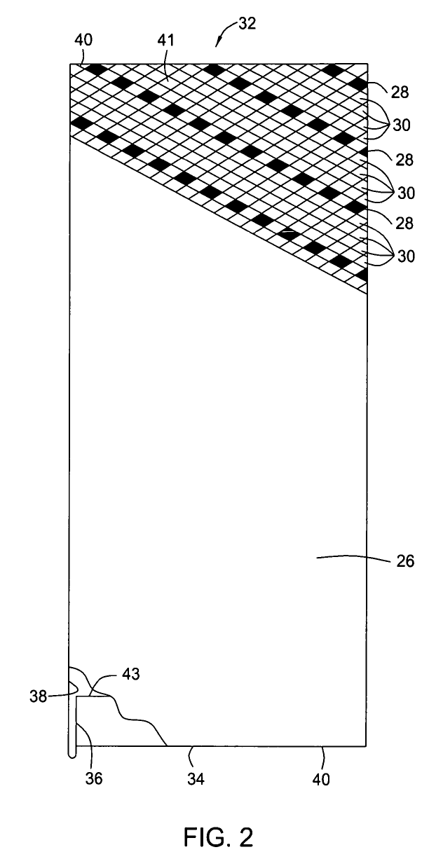

Referring now to FIG. 2, the structure of the integral stent graft 10 is shown in greater detail. Stent graft 10 includes integral body portion 26, (multiple such portions may be provided to form as bifurcated stent graft 10) formed of the interconnection, such as by weaving, braiding, or the like, of individual wires 28 and threads or filaments of fabric 30. For ease of illustration, the general configuration of body portion 26 is shown, but only a portion of the wires 28 and filaments of fabric 30 are shown, it being understood that the pattern shown extends over the entire body portion 26. Where stent graft 10 is the bifurcated stent graft shown in FIG. 1, each of trunk portion 24 and branched portions 20, 22 thereof is comprised of an individual tubular body portion 26, having opposed ends 32, 34, which are provided by folding in the tubular body 26 at the opposed ends 32, 34, to provide a circumferential flap 36 (shown in partial cutaway at end 34 only), which terminates at fabric cut 43 where the tubular body is cut to length, and is affixed to the interior surface 38 of the tubular body portion 26 such as by sewing, heat or laser welding, and the like. (Graft portion 24 would have two open second ends 34, for receipt of two branched portions 20, 22 therein). By folding flap 36 inwardly of the tubular body portion 26 and affixing it thereto, the continuous edge surface 40 is provided at the opposed ends 32, 34 of the tubular body portion 26, and that portion of the tubular body most liable to fraying, i.e. the ends 32, 34 thereof, is secured within the body portion 26. Alternately, the ends 32, 34 may be terminated without a flap, such that heat is applied to the ends 32, 34 to cause slight re-melting, followed by reformation in solid form, of the fabric, which results in a sealed end 32 or 34 where the fabric 30 is configured of polyester or the like. Additionally, flap 36 may be folded over the outer portion of body portion 26, and secured thereto in the same manner as if it were folded against the inner portion thereof.

As is also shown in FIG. 2, in one embodiment of the invention the structure of the stent graft body portion 26 is provided by the interbraiding of individual wires 28 and individual filaments of the fabric 30, extending in a helical fashion about the circumference of the integral stent graft body portion 26. In completed form, as shown in FIG. 2, there results a tubular structure, having a fluid barrier structure provided essentially by the interbraided fabric 30 filaments, as well as a structural component, primarily provided by the interbraided wires 28. To provide the interbraided structure, a portion of the wires 28 and filaments of fabric 30 extend with a left hand pitch, from one end 32 of the tubular body to the other end 34 thereof, and second plurality of the wirers 28 and filaments of fabric 30 extends between ends 32, 34 with a right hand pitch. Thus, both the wires 28, and the filaments of fabric 30, form an intersecting diamond pattern 41 along the length of the tubular body portion 26.

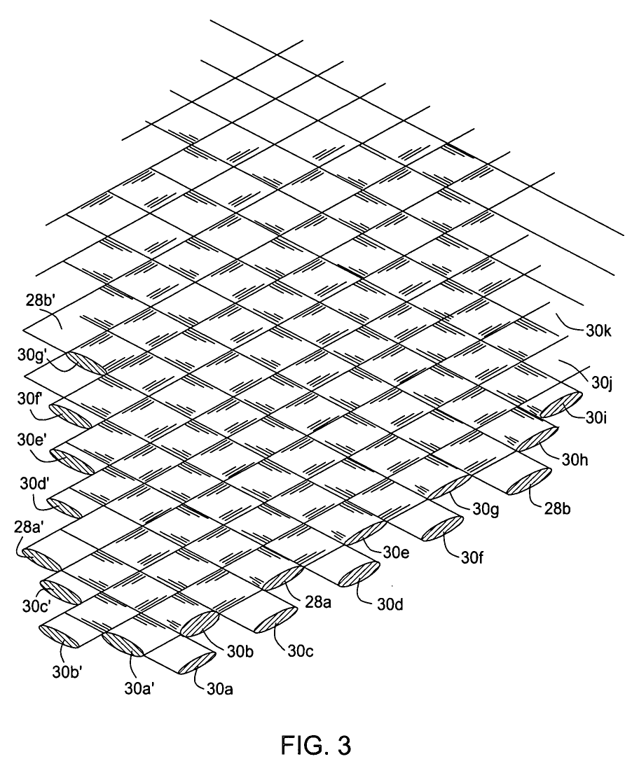

Referring now to FIG. 3, there is shown an enlarged view of a portion of the stent graft of FIG. 2, showing in greater detail the braided structure of the stent graft 10. As shown, body portion 26 is comprised of individual groups of filaments 30a, 30b, 30c, 30j, et. seq., interspersed with wires 28a, 28b, 28c, et. seq., forming a portion of the body portion 26 and disposed in a right hand direction, as well as corresponding primes thereof (e.g., filaments of fabric 30a′, 30b′, et. seq., and wires 28a′, 28b′, et. seq.), forming the remainder of body portion 26 and running in a generally left hand direction. Each filament 30a, 30a′, et. seq as well as wires 28a, 28a′, et. seq., preferably extends the entire length of body portion 26, i.e., from end 32 to end 34. Filaments 30a, et. seq. and wires 28a, et seq., are interbraided, such that each filament of fabric 30a et. seq. or wire 28a et. seq. disposed in a right hand direction continuously passes over and under filaments of fabric 30a′ et. seq. or wires 28a′, et. seq. as they are encountered between opposed ends 32, 34 of the body portion. For example, as shown in FIG. 3, filament 30a, which passed in a right hand direction down the length of tubular body 26, is shown passing under filament 30a′, over filament of fabric 30b′, under filament of fabric 30c′ etc., all of which pass along body portion 26 in a left hand direction. Likewise, filament 30b, located next to filament 30a, passes over filament 30a′, under filament 30b′, etc. Thus, each right hand direction filament 30a, et seq., as well as every right hand direction wire 28a, et. seq., sequentially travels over and then under each successive filament 30a′, 30b′ et. seq. and wire 28a′, 28b′, et seq. encountered, to form a continuous, tightly interdigitated fabric, having sealing capability as well as structural rigidity in use.

Stent graft of the form shown in, and described with respect to, FIGS. 2 and 3, is preferably manufactured on a braiding machine having a multitude of spindles and capable of interlacing the right hand and left hand strings to form a braided tubular structure. Preferably, the braider is a maypole style machine, having a multitude of carriers, a portion of such carriers dispensing the filaments of fabric 30, and a further plurality dispensing the wire 28. For example, a ninety-six-spindle maypole style-braiding machine may be used to create the body portion 26, wherein twenty of the spindles dispense the wire 28, and seventy-six of the spindles 30 dispense the filaments of fabric 30. One-half of the wires 28 (ten) are dispensed to travel in a left hand fashion for fabrication, and one half (ten) in a right hand fashion for fabrication. Likewise one-half (thirty-eight) of the filaments of fabric 30 are dispensed to travel in a left hand fashion, the other thirty-eight are dispensed to travel in a right hand fashion. The wires 28 are preferably evenly circumferentially spaced such that, in this embodiment, the wires 28 each have either 4 or 5 of filaments of fiber 30 spaced between adjacent wires 28. Wire is preferably of approximately 0.025 mm in diameter, and filaments are of a similar size. Wire 28 is preferably comprised of a Nitinol material, which has shape memory characteristics. Filaments of fabric 30 are preferably polyester or other known biocompatible material, which, where braided with wire 28 as discussed herein, can form a fluid barrier tube. Additionally, wire 28 may be previously interbraided or bundled, such that each wire location of FIGS. 2 and 3 are replaced with a bundle of 2 to 5 wires. In such configuration, the wire bundle would have an effective diameter on the order of 0.05 to 0.1 .mm, and the filaments of fiber may be of smaller or equal size. Additionally, to form a stent graft or the individual elements of a bifurcated stent graft 10, a length of braided tube greater than the necessary length of the body portion may be provided, and individual body portions 26 cut and configured (such as by sewing flaps 36 into position) therefrom. Alternatively, body portions 26 may be individually prepared by the braiding machine. Further, the diameter of the resulting body portion 26 may be varied, to provide stent grafts of gradations in size or to a specific patient need.

The resulting tubular structure is used to form the trunk portion 24 and branch portions 20, 22. Preferably, two individual sections may be formed, one comprising the trunk portion 24 and branch section 20 integrally formed, and a second section forming branch section 22. Thus, trunk portion 24 extends integrally to form branch section 20, and an aperture 80 is formed adjacent the transition from the trunk portion 24 to the branch section 20, into which the second branch section 22 is positioned when the stent graft is deployed in the aorta or other blood vessel. This configuration may be provided by forming trunk section 24 with two open ends, and sewing, using an adhesive or interbraiding the ends thereof, or otherwise attaching the branch section 20 to trunk section 24 at one of the openings. Thus, the first portion of the stent graft to be deployed will include an integral trunk and branch section, to which a second branch section 22 may be deployed as previously described herein. Alternatively, separate trunk portions 24 and branch sections 20, 22 may be provided, such that trunk portion 24 is first deployed in the aorta, and each of the branch sections are deployed within the individual openings therein.

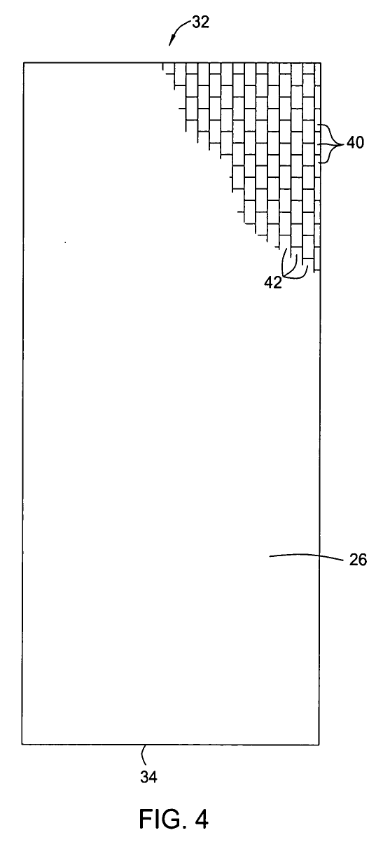

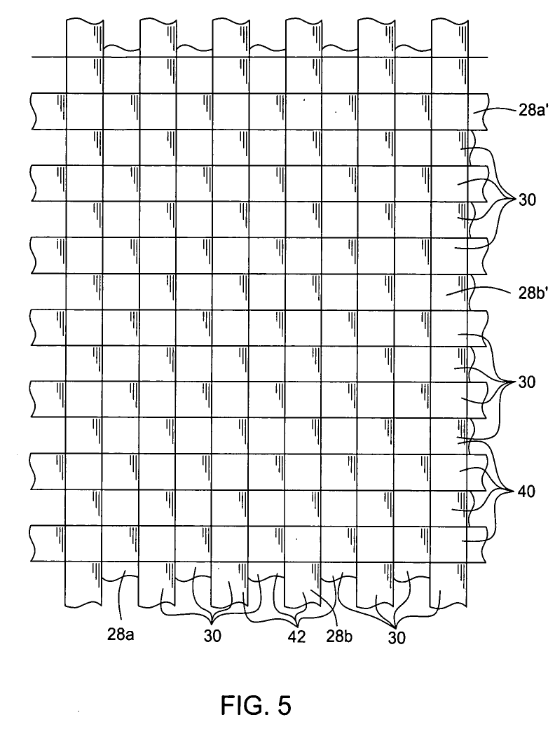

Referring now to FIGS. 4 and 5, there is shown an alternative style of stent graft 10, wherein tubular body 26 is woven, rather than braided. In this embodiment, a continuous tubular body portion 26 includes a plurality of hoop elements 40 which extend in a generally circular path about the circumference of the body portion, and a plurality of generally longitudinal span elements 42, which extend the length of the stent graft body portion from end 32 to end 34. As with the braided embodiment of the body portion previously described, span elements 42 and hoop elements 40 of the body portion 26 are preferably wires 28 and filaments of fabric 30, which are interdigitated or weaved, to form a barrier to liquid sleeve or body portion 26 having integral support therein. Thus, hoop elements 40 are provided as a plurality of filaments of fabric 30, interdigited with the span elements 42 and interspersed with wires 28. Preferably, one wire 28 is provided for every three to five filaments making up the hoop elements 40. Hoop elements 40 are sized, in length, such that the individual filaments 30 or wires 28 are slightly longer than the circumference of the resulting woven structure, such that an offset (not shown) is provided and the hoop elements 40 may extend at least one circumference of the resulting body portion 26. Preferably, hoop elements 40 extend two or more circumferences of the woven structure, and may extend, in helical fashion, as long the entire length of body portion from end 32 to end 34. Span elements 42 are likewise comprised of interspersed wires 28 and filaments 30 in a one wire 28 to three to five filament of fabric ratio, and extend a distance greater than the length from end 32 to end 34. The ends of the span elements 42 may be reversed and integrally woven into the interdigited hoop and span elements, as is well know in the art, to provide a relatively non-frayable end 32 or 34.

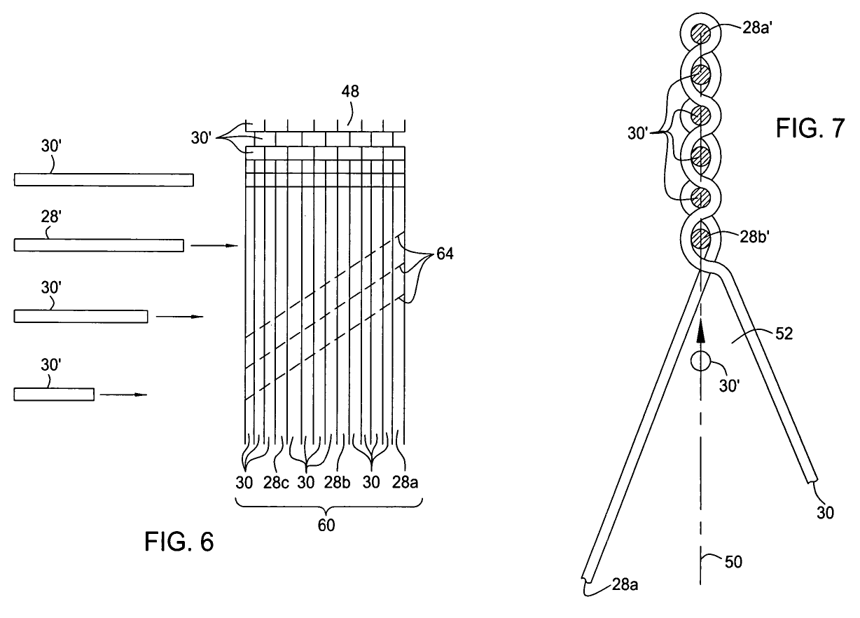

To create woven stent graft body 26, a weaving machine or loom capable of providing a tubular woven structure is provided with wires 28 and filaments 30. A plurality of longitudinally disposed wires 28 and filaments of fabric 30 are aligned, longitudinally, as shown in FIG. 6, such that adjacent wires 28a, 28b are disposed with a plurality of filaments 30 therebetween, preferably between three and five. The longitudinally aligned wires 28 and filaments of fabric 30, extend from a partially formed woven cylinder 48 (only partially shown in FIG. 6) and thus form a loose cylindrical configuration of individual strands of wire 28 and filaments of fabric 30. A plurality of circumferential wires 28′ and filaments 30′ are then interdigited with the individual longitudinally disposed wires 28 and filaments 30, and racked into a tightly woven mesh as shown in detail in FIG. 7. Essentially, as is shown in FIG. 7, in operation individual strands of the longitudinal elements (one of wires 28 and filaments 30 shown) are alternately actuated inwardly and outwardly of a baseline, true cylindrical position 50, such that each adjacent strand (wire 28 or filament of fabric 30) of the plurality of wires 28 and filaments of fabric 30 is oppositely positioned, and the individual strands of the circumferential components (wire 28′ and filaments 30″) are threaded into the gap 52 between the adjacent strands of the longitudinal elements and then, once positioned circumferentially thereabout, are racked into intimate contact with the longitudinal strands adjacent to the last circumferential strand previously racked. After each strand (wire 28′ or filament of fabric 30′) of the circumferential components is so positioned, the position of the individual strands of the longitudinal components is switched, i.e., if wire 28 had been biased inwardly it is now biased outwardly, and the next strand of the circumferential elements is placed into the gap. Preferably, circumferential strands are provided in the same ratio and spacing of wires to filament as the longitudinal elements.

Referring again to FIG. 5, the longitudinal and circumferential strands result in a pattern whereby longitudinal wires 28a, 28b, et. seq. regularly cross over and under circumferential wires 28a′, 28b′, et. seq., resulting in a regular rectangular (where equal spacing of the wires is used for both longitudinal and circumferential wires, a square) pattern or outline provided by the crossing wires. However, it is specifically contemplated herein that other wire crossing outlines may be produced by weaving. For example, a parallelogram or diamond shaped pattern may be provided, such as by modifying the longitudinal strands such that the lower ends thereof are rotationally misaligned (as shown by arrow 60 in FIG. 6) resulting in a canted of offset wire position, and further by loading the circumferential strands in a tilted fashion, as shown by dashed lines 64 representing the orientation of the circumferential strands (wires 28 and filaments of fabric 30) as they are positioned for weaving. The result is a diamond patent between the wires, as the wires 28, 28′ cross each other at a non-perpendicular angle, preferably an angle of between 30 and 45 degrees. Wires 28a, 28b et. seq and, 28a′, 28b′ et. seq. are preferably composed of Nitinol, a shape memory material, and preferably have a diameter on the order of 0.025 mm diameter. The filaments of fabric 30a, 30b et. seq. and, 30a′, 30b′, et. seq. are preferably composed of polyester, likewise having a diameter on the order of 0.025 mm. The wires may also be placed into the body portion 26 as strands or bundles of wires 28, such that 2 to 5 individual wires may be bundled, braided or intertwined and located in the body portion in the place of individual wires 28, 28′.

After formation of the tubular body portion 26 by weaving, the opposed ends 32, 34 are folded over to form a creased end, and these folded over portions are secured to the tubular body. Preferably, these folded ends are interweaved into the tubular structure forming body portion 26, and thus an integral end seam of high structural integrity is formed. However, the folded over ends may likewise be sewn, sonic or heat welded, glued or otherwise secured to body portion 26. As with the embodiment of the stent graft using braiding, the body portions hereof may be cut from a continuous tubular length of woven wires 28 and filaments of fabric 30, or individual discreet woven body portions 28 may be provided.

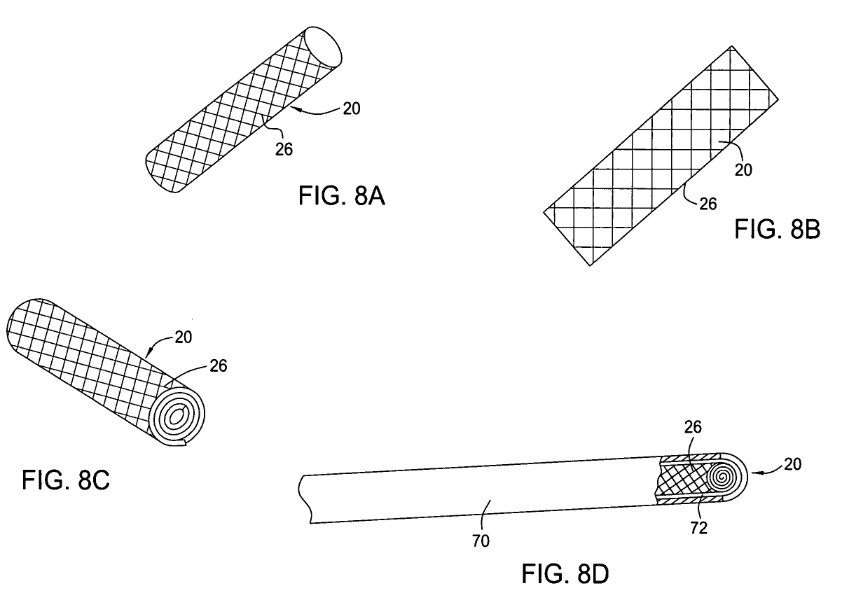

Referring now to FIGS. 8a to 8d, there is shown the methodology for preparing a stent graft 10 of the present invention for delivery. Body portion 26 is shown in FIG. 8a in its fully expanded, as manufactured size, which is preferably slightly larger in diameter than the body lumen in which it will be placed. Body portion 26 in this particular embodiment is a branch portion 20 of a bifurcated stent graft 10 as shown in FIG. 1. Branch portion 20 is then placed in liquid nitrogen, or otherwise cooled to very low temperatures (liquid nitrogen having a temperature in the range of minus 196 degrees Celsius). Once cooled, the body portion 26 forming branch portion 20 is flattened to the configuration shown in FIG. 8b. Body portion 26 forming branch portions 20 is then rolled, as shown in FIG. 8c, such that it has a resulting diameter on the order of up to one cm. The resulting rolled configuration of body portion 26 forming branch portion 20 is then inserted into the open end 72 of a catheter 70 as shown in FIG. 8d. The body portion 26 forming branch portion 20 is allowed to regain room temperature, at which time it is ready for deployment in a body lumen. To deploy the body portion 26 forming branch portion 20, the catheter 70 is inserted into a blood vessel of a patient (not shown), and the end thereof holding branch portion is pushed into a position adjacent to a previously deployed trunk portion 24 spanning an aneurismal aorta site. Branch portion 26 is then pushed out of the catheter 70, to a position connecting at one end thereof to the open end of the trunk portion 24, and at the other end thereof to span the aneurismal location and engage healthy blood vessel wall, and thereby complete assembly of the stent graft 10 in place. Where the aneurysm is located such that a single tubular body may effectively span and seal off the aneurysmal sac 18, body portion 26 forms the entire stent graft.

Once the body portion 26 forming branch portion 20 leaves the catheter, the shape memory wire 28 material will expand to its as manufactured configuration, i.e., the position prior to being cooled, flattened and rolled, being previously prevented from doing so by the wall of the catheter 70 in which it was placed. The integral nature of the body portion (s) of the stent graft 10 ensures that the filaments of fabric 30 providing the graft material will be intimately biased, by the wires 28, into sealing engagement with the lumen wall (or the interior of the trunk portion 24 where the stent graft 10 is a bifurcated structure), because only a few such filaments of fabric 30, on the order of three to five, are preferably positioned between each adjacent wire 28 forming the stent portion of the integral stent graft 10. However, other ratios of wires 28 to filaments of fabric 30 may be used, so long as adequate structural rigidity and sealing properties are maintained. Additionally, the stent graft 10 provides a body portion 26 as a smooth bodied, easily deployable unit, without seams, sewing or other mechanisms to attach the graft to the stent. Further, the stent graft 10 of the present invention is easily manufacturable in discrete, patient ready, sizes, tailored to the individual lumen properties of a particular patient (aneurysm diameter, etc). Further, the integral stent graft 10 may be configured from very small diameters, on the order of a quarter inch, to diameter on the order of several inches, if needed.

I claim:

1. A stent graft, comprising a unitary body having interdigitated structural and fluid barrier elements therein.

2. The stent graft of claim 1, wherein the unitary tubular body is formed by weaving said structural and said fluid barrier elements together.

3. The stent graft of claim 1, wherein braiding forms said tubular body said structural elements and fluid barrier elements together.

4. The stent graft of claim 1, wherein said structural elements include a shape memory element.

5. The stent graft of claim 4, wherein said shape memory element is nitinol.

6. The stent graft of claim 5, wherein said tubular body is flattened and rolled at a temperature other than the stent graft delivery temperature and delivered to a treatment location is said rolled state.

7. The stent graft of claim 1, wherein said stent graft has a first shape and a second, altered shape; and
said stent graft is delivered to a treatment site in said altered shape, and, upon delivery, said stent graft regains its first shape.

8. The stent graft of claim 7, wherein said first shape is generally tubular.

9. The stent graft of claim 1, wherein said fluid barrier elements include polyester fabric.

10. The stent graft of claim 1, wherein said unitary body forms a portion of a bifurcated stent graft.

11. The stent graft of claim 1, wherein said unitary body includes opposed open ends, and the portion of said body adjacent said opposed open ends is folded back and secured to said body.

12. The stent graft of claim 11, wherein said the portion of said body adjacent said opposed open ends which is folded back is secured to said body by interweaving.

13. A method of providing an intraluminal exclusion device, comprising the steps of:
providing a plurality of structural elements;
providing a plurality of fluid barrier elements; and
interdigitating said structural elements and said fluid barrier elements to form a unitary body.

14. The method of claim 13, wherein the step of interdigitating includes the step of interweaving the structural elements and the fluid barrier elements.

15. The method of claim 13, wherein the step of interdigitating includes the step of interbraiding the structural elements and the fluid barrier elements.

16. The method of claim 13, wherein the structural elements and the fluid barrier elements are generally cylindrical in form.

17. The method of claim 16, wherein the number of fluid barrier elements in the exclusion device exceed the number of structural elements by a factor of three to five.

18. The method of claim 16, wherein the structural elements are wires.

19. The method of claim 16, wherein the structural elements are bundles of wires.

20. The method of claim 16, wherein the fluid barrier elements are filaments of fabric.

21. The method of claim 20, wherein the fabric is polyester.

22. The method of claim 13, further including the steps of:
cooling the exclusion device;
flattening the cooled exclusion device;
rolling the cooled and flattened exclusion device into a cylinder;
inserting the cylinder into a catheter;
delivering the cylinder, via the catheter, to a body lumen location;
disposing the exclusion device within the body lumen; and
allowing the exclusion device to expand to its pre-cooled configuration and engage the interior wall of the body lumen at least one end thereof.

23. The method of claim 22, wherein the exclusion device is a stent graft and the body lumen location is an aneurismal blood vessel.
